(19) United States
(12) Patent Application Publication
(10) Pub. No.: US 2003/0069557 A1
(43) Pub. Date: Apr. 10, 2003

(54) Absorbent garment with integrated elastiziced connector tabs and waistline

(76) Inventors: Stacy Driskell, Alpharetta, GA (US); Sunita Pargass, Norcross, GA (US); Joseph Vergona, Swanee, GA (US)

Correspondence Address: Christopher C. Campbell, Esq., Hunton & Williams, Suite 1200, 1900 K Street, NW, Washington, DC, 20006-1109, US

(21) Appl. No.: 09/972,868

(22) Filed: Oct. 10, 2001

(51) Int. Cl. A61F 13/15

(52) U.S. Cl. 604/385.3; 604/386

(57) ABSTRACT

An absorbent garment includes a chassis comprised of a moisture impervious outer layer, a moisture pervious inner layer, and an absorbent layer sandwiched between the inner and outer layers. The garment includes one or more elasticized connector tabs attached to the chassis such that part or all of one waist portion of the garment is expandable. The gripping connectors, attached to the distal ends of the connector tabs, interlock with a target connector located near the opposite wait edge of the garment. The waist, made expandable by the integrated elastic connector tabs, conforms to the contours of the user, increasing comfort and improving leak resistance. The connector tabs may be constructed from nonwoven material sandwiching an elastic element or elements.

### BACKGROUND OF THE INVENTION

1. Field of the Invention

The invention relates to the field of absorbent garments in general, and more particularly to an absorbent article having an elastic fastener that is integrated into the body of the garment to create a stretchable waistband for improved fit and comfort, and for reduced leakage.

2. Description of Related Art

Disposable absorbent garments (“garments”) such as infant diapers or training pants, adult incontinence products, and other such products are well-known in the art. Typically, the chassis of such garments comprise a liquid-permeable body-contacting liner sheet (or “top sheet”), a liquid-impermeable backing sheet (or “back sheet”), and a moisture-absorbent core (or “absorbent core”) that is generally disposed between the liquid-permeable liner sheet and the liquid-impermeable backing sheet. These garments oftentimes include additional features, such as elastic waist bands, elastic leg bands, and stretchable side panels. In addition, decorative graphics are often incorporated into the disposable absorbent article for aesthetic and functional purposes.

Despite advances in the field of absorbent articles, urinary and bowel movement (“BM”) leakage from absorbent articles continues to present a formidable challenge to absorbent article manufacturers and others skilled in the art. Leakage of BM and urine from absorbent articles occurs when the absorbent cores of such garments cannot absorb body exudates fast enough, resulting in a lateral flow of the excreted matter toward the sides of the garment. Manufacturers rely on the leg elastic gathers (used to secure the garment to the legs of the wearer), internal baffles, and other structures to prevent body exudates from leaking out of the garment in the region surrounding the wearer's legs.

Urine and BM also can leak out of the waistband area. For example, urine and BM may approach and leak out of the waistband when the user is placed on his or her back to change the garment, when the user leans forward, sits backwards, lies down, or crawls. To prevent such leakage, it is desirable to have a good seal between the garment and the user around the entire perimeter of the leg holes and the waistline. This problem has been addressed by several inventions, such as the invention disclosed in Schaar U.S. Pat. No. 3,995,640, which discloses a garment with an elastic band incorporated into the waistline of the garment's chassis. Other patents disclose similar constructions to achieve an elasticized waistband. For example, Sciaraffa, et al. U.S. Pat. No. 4,381,781 and Wood et al. U.S. Pat. No. 4,857,067 both disclose garments with waistbands that are elasticized by integrating elastic material into the waistband. Other inventions, such as Jacobs, U.S. Pat. No. 3,800,796, disclose an inelastic garment chassis that uses elastic connection straps to hold the garment on the user.

Another problem absorbent garment manufacturers face is that manufacturers must produce a broad range of garment sizes to accommodate the great variety of user sizes. A garment that is too large or small for the user may be uncomfortable, and typically is prone to leakage, premature release, and other problems. To solve these problems, traditional garments have been made in a range of sizes to better fit the various sizes of users. This solution is incomplete in that it increases the manufacturing cost of the garments because more machinery (or more complex machinery) and processing steps are required to produce the required range of garment sizes.

Despite efforts made by those skilled in the art, leakage around the waistband and the need for a broad range of garment sizes still remain problems. These and other problems are addressed by the current invention.

### SUMMARY OF THE INVENTION

In response to the difficulties encountered in the prior art, a disposable absorbent garment is disclosed having integrated elasticized connectors and waistline.

The garment of an embodiment of the invention comprises an outer layer made from a liquid impervious material, a liquid pervious inner layer, and an absorbent layer sandwiched between these layers. The outer layer preferably is made from a “breathable” vapor- or air-permeable material. The garment, as depicted herein, preferably is comprised of single, continuous outer, inner, and absorbent layers forming the waist and crotch regions. It should be evident, however, that the present invention may be made from separately manufactured front and rear waist portions joined at the crotch.

The garment includes a pair of elasticized connector tabs attached to the outer layer of the garment along the side edges and near one waist edge of the garment. Alternatively, connector tabs may be attached to the interior layer, or sandwiched between the outer and inner layers. The interior end of the connector tabs are located well inboard of the lateral edge of the garment, and the exterior ends of the connector tabs extend beyond the lateral edge of the garment. Thus located, the connector tab overlaps the chassis of the garment over a substantial area, within which area the connector tab is operably attached to the chassis, and extends beyond the edge of the garment. As understood herein, “chassis” refers to the main body of the garment, including the outer layer, inner layer, and all attachments thereto, but excluding the connector tabs and the gripping connectors (which are permanently attached to the connector tabs).

A gripping connector is attached to the exterior end of each connector tab. The gripping connector is used to hold the connector tab to the opposite waist portion of the garment by using a mechanically interlocking interface between the gripping connector and target connectors located at or near the opposite waist edge of the chassis.

In another embodiment of the invention, a single elasticized connector tab, which spans the entire width of the garment is attached near one waist edge of the chassis. In this embodiment, the connector tab has a gripping connector at both ends.

Furthermore, the elasticized connector tabs are attached to the chassis of the garment in their extended position, so that when the connector tabs are in their contracted state both the connector tabs and the portion of the chassis to which the connector tabs are connected are shirred or gathered. When the garment is worn, both the connector tabs and the chassis of the garment extend to conform to the user's body.

One advantage of an embodiment of the invention is that by incorporating the elastic connector tab into the chassis of the garment, a greater amount of flexibility is added to the garment. A garment constructed in this manner can accommodate a greater range of body sizes. For example, if the elastic connector tabs are incorporated into an existing garment, the garment will be able to operate on smaller bodies because the elastic straps will gather up the slack that would exist if a conventional garment were being used. The garment thus equipped will also be able to work on larger bodies because the elastic straps, when fully extended, can reach farther than the connecting straps used in a conventional garment, to secure the garment to the user.

Another advantage of embodiments of the invention is that the shirred waistline and connector tab add flexibility around the hip and leg of the user, as opposed to the user's back and stomach. This location helps make the garment flexible where it is most required, as a user typically exhibits a more complex rotational motion in the leg and hip area than in the back, which is generally a simple bending motion. By increasing the flexibility at the user's joints, the garment can grip the user more effectively in various user positions, thereby resisting urine and BM leakage. Such a garment also fits the user more comfortably than a garment with less flexibility in these areas.

Yet another advantage of embodiments of the invention is that the elasticized connector tab can be constructed such that the garment does not require an additional waist elastic to be incorporated in the garment, although a waist elastic can still be used to improve garment comfort and sealing. Another advantage of an embodiment of the invention is the ease with which an elastic connector tab can be joined to a conventional garment chassis. This reduces the cost of manufacturing the garment.

These and other objects, features and advantages of the invention will be apparent through the detailed description of the preferred embodiments and the drawings attached hereto. It also is to be understood that both the foregoing general description and the following detailed description are exemplary and explanatory and are not restrictive of the scope of the invention.

### BRIEF DESCRIPTION OF THE DRAWINGS

The invention will be described with respect to the accompanying drawings, in which like elements are referenced with like numbers.

FIG. 1 is a cutaway plan view of one embodiment of a garment of the invention;

FIG. 2 is an isometric view of one embodiment of a connector tab in the fully-extended position;

FIG. 3 is an isometric view of another embodiment of a connector tab in the fully-extended position;

FIG. 4 is a partially-cutaway plan view of one embodiment of a connector tab integrated with the body of a disposable absorbent undergarment;

FIG. 5 is a partially-cutaway plan view of an embodiment of the invention in which there is a single connector tab spanning the entire width of the garment.

FIG. 6 is a cross-sectional view of an embodiment of the invention in which the connector tab is located adjacent to the outer layer of a disposable absorbent undergarment and the connector tab is in the fully-extended position;

FIG. 7 is a cross-sectional view of an embodiment of the invention in which the connector tab is located adjacent to the inner layer of a disposable absorbent undergarment and the connector tab is in the fully-extended position;

FIG. 8 is a cross-sectional view of an embodiment of the invention in which the connector tab is sandwiched between the inner layer and outer layer of a disposable absorbent undergarment and the connector tab is in the fully-extended position;

FIG. 9 is a cross-sectional view of the connector tab as depicted in FIG. 5 with the connector tab in the fully-relaxed position;

FIG. 10a is a drawing of a connector tab integrated with the body of a garment with the elastic in the relaxed position;

FIG. 10b is a drawing of a connector tab integrated with the body of a garment with the elastic in the extended position;

FIG. 11a is a drawing of the second waist edge portion of an embodiment of the invention showing the garment connector tabs and waist elastic in the extended position;

FIG. 11b is a drawing of the second waist edge portion of an embodiment of the invention showing the garment connector tabs and waist elastic in the contracted position;

FIG. 12a is a drawing of the second waist edge portion of a comparative garment showing the waist elastic in the extended position; and

FIG. 12b is a drawing of the second waist edge portion of a comparative garment showing the waist elastic in the contracted position.

### DETAILED DESCRIPTION OF THE PREFERRED EMBODIMENTS

“Garment,” as used herein, refers to articles and garments that absorb and contain body exudates, and more specifically refers to articles and garments that are placed against or in proximity to the body of the wearer to absorb and contain the various exudates discharged from the user's body. A non-exhaustive list of examples of “absorbent articles” and garments includes diapers, diaper covers, disposable diapers, training pants, feminine hygiene products, and adult incontinence products. The invention can be used with all of the foregoing classes of absorbent articles and garments, without limitation, whether disposable or otherwise. Furthermore, the invention will be understood to encompass, without limitation, all classes and types of absorbent articles and garments, including those described above.

Although the various embodiments of the invention are described in the context of a diaper, it is readily apparent and understood that this is not intended to limit the invention.

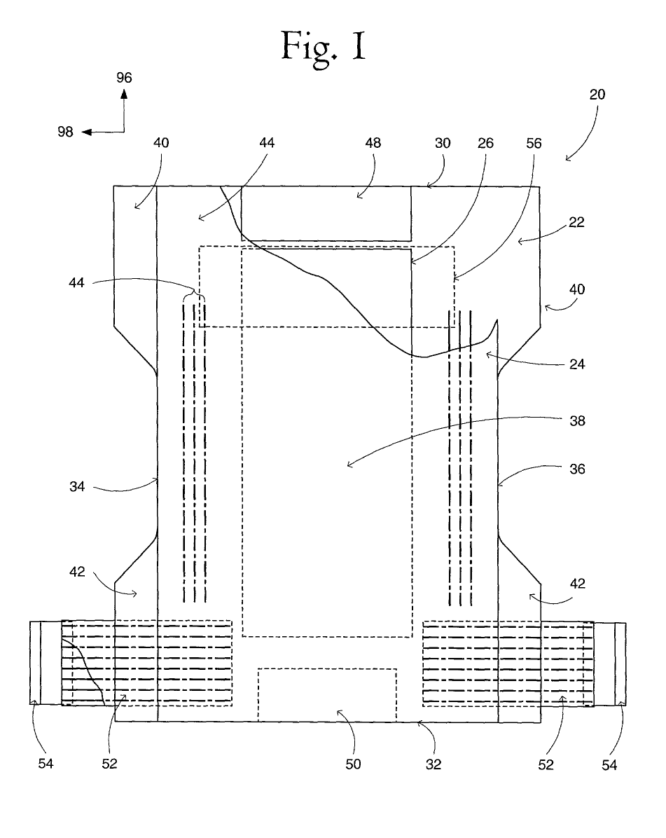

FIG. 1 illustrates an embodiment of a garment 20 having a longitudinal dimension 96 and a lateral dimension 98. The garment has a moisture impervious outer sheet 22, often referred to as a backsheet, and a moisture pervious body-contacting inner layer or sheet 24, often referred to as a top sheet. A moisture absorbent fluff filler pad layer or core 26 is sandwiched and encased between the inner sheet 24 and the outer sheet 22. The encasement of the garment may be accomplished by a variety of means, well known in the art, including bonding the layers together by a plurality of fine hot melt adhesive lines, bonding the layers using construction adhesives, or using ultrasonic bonding to join the layers. For clarity, FIG. 1 illustrates the garment with all of its elastic components, which are described herein, in their fully extended positions. When the elastic components are in their relaxed or contracted positions, the garment will have shirred or wrinkled areas that can expand when force is applied in the plane of the garment.

The manufacture of the chassis of such a garment is well-known in the art. For example, the moisture impervious outer layer 22 may comprised of a thin thermoplastic material, such as a pigmented polyethylene film having a thickness in the range of 0.02-0.04 mm. The outer layer 22 also may be a layered material comprised of one or more layers of meltblown polypropylene or meltblown polyethylene, sandwiched between layers of spun-bonded material.

The moisture permeable inner layer 24 typically is comprised of a carded polyester fiber with a latex binder or of a spun-bonded polypropylene having continuous fibers and thermally bonded by patterned calendar rolls. The moisture permeable inner layer 24 may be treated over its entire surface to render it hydrophilic. The inner layer 24 also may be zone-treated with a surfactant to make it hydrophilic only in certain target areas. The surface treatment can be accomplished by a variety of means well known in the art.

The moisture absorbent layer or core 26 may be comprised of any absorbent material or materials known in the art, such as, but not limited to, wood fibers or other fibers such as chemical wood pulp, or any other suitable liquid absorbing material such as commercially available fluff pulp or of a fluffed bleached kraft softwood pulp. The fluff component may also be mixed with distributed particles of superabsorbent material, with the resulting core 26 being preferably surrounded by a tissue over-wrap, or other material, to contain the superabsorbent particles. The moisture absorbent layer 26 may extend for the entire length and width of the garment, but alternatively it may extend only partially along the length and width of the garment.

The garment 20 usually has a first waist edge 30, which may lie along the front waist of a wearer, and an opposed second waist edge 32, which may lie along the waist at the wearer's back. Two opposed side margins 34, 36 define leg encircling openings when the garment is fitted on a wearer. Thus, with the first waist edge 30 to the front of a wearer, side margin 34 is the left leg side margin and side margin 36 is the right leg side margin. Lying between the opposed side margins 34, 36 is a crotch region 38, which is central to the garment.

As illustrated in the embodiment of FIG. 1, the outer layer 22 may extend outwardly from each of the opposed side margins 34, 36 along the first and second laterally extending waist edges 30, 32. In this manner, a pair of first waist extensions or “ears” 40 are formed extending from each side margin along the first waist edge 30. A pair of second waist extensions or “ears” 42 are formed extending from each side margin along the second waist edge 32. The inner layer 24 may extend partially or completely over the waist extensions 40, 42 or it may terminate just beyond the outer boundary of the absorbent core 26 along an outer boundary 44. The outer boundary 44 is the region in which the inner sheet 24 is bonded or joined to the outer sheet 22 to encase the absorbent core 26.

Thus, the sandwiched layers of the garment 20 as illustrated in FIG. 1, may have an hourglass shape. The top and bottom of the hourglass shape form first and second waist portions of the garment, with the first waist portion comprising each of the first waist extensions 40 and the area of the diaper therebetween. The second waist portion comprises the second waist extensions 42 and the region of the garment along the second waist edge 32 between extensions 42. The opposed sides of the hourglass shape form the opposed side margins 34, 36. The crotch portion of the garment 38 is central to the hourglass shape, and has a narrower width than the waist portions. Alternatively, the garment 20 may have a rectangular shape, with no waist extensions 40, 42. As yet another alternative, the garment 20 may have a “T” shape, having only a pair of first waist extensions 40, or a pair of second waist extensions 42.

The leg openings defined by the opposed side margins 34 and 36 preferably are gathered and elasticized to create a better fitting garment that is more capable of retaining bodily wastes. There are a variety of methods for providing elasticized leg openings, well known in the art, by incorporating elastic materials into the garment 20 between the outer layer 22 and the inner layer 24 along each of the opposed side margins 34, 36. The elastic material can be in the form of longitudinal strands 22, webbing, strips, or any other functional configuration. Any suitable elastic material may be used, such as rubber, elastic foams, Spandex (which is commercially available from E. I. DuPont Nemours & Company, a business with its corporate headquarters in Wilmington, Del.), or LYCRA® (also available from E. I. DuPont Nemours & Company). In one embodiment of the invention, the elastic material is expanded and then attached to the garment using elastic adhesives, such that when the elastic relaxes or contracts the material of the garment forms expandable shirrs or gathers in the side margins 34 and 36. In another embodiment of the invention a heat-shrinkable elastomeric material, of the type that is relatively unstable and inelastic when unshrunk, and relatively stable and elastic when shrunk, is incorporated into the garment and subsequently heat shrunk to form expandable shirrs or gathers in the side margins 34 and 36.

In one embodiment, the invention includes a first waist elastic 48 in the boundary area 44 adjacent to the first waist edge 30, and a second waist elastic 50 in the boundary area 44 adjacent to the second waist edge 32. The waist elastics 48 and 50 preferably are formed by placing an elastic material, such as an expandable foam or a heat-shrinkable elastomeric material, between the outer layer 22 and the inner layer 24. Such a construction may be done as described above regarding the elasticized leg openings. Although the garment 20 illustrated in FIG. 1 has both a first waist elastic 48 and a second waist elastic 50, it is anticipated that an embodiment of the invention can have a waist elastic, as described above, in only one of the waist edges 30, 32 or in neither of the waist edges 30, 32.

The garment 20 preferably has two connector tabs 52 (“tabs”) protruding laterally from the second waist extensions 42 near the second waist edge 32. Each tab 52 has a gripping connector 54 located at or near its laterally outboard end. The garment is fitted to the wearer by attaching the gripping connectors 54 to a target connector 56 located on the outer sheet 22 near the first waist edge 30. Although the embodiment of the invention depicted in FIG. 1 has a single target connector 56, it should be apparent that the invention could also be practiced using a plurality of target connectors attached to the outer layer 22, the inner layer 24, or both. Furthermore, it should be clear that embodiments of the invention may be practiced with the connector tabs 52 preferably located near the first waist edge 30, and the target connector located near the second waist edge 32.

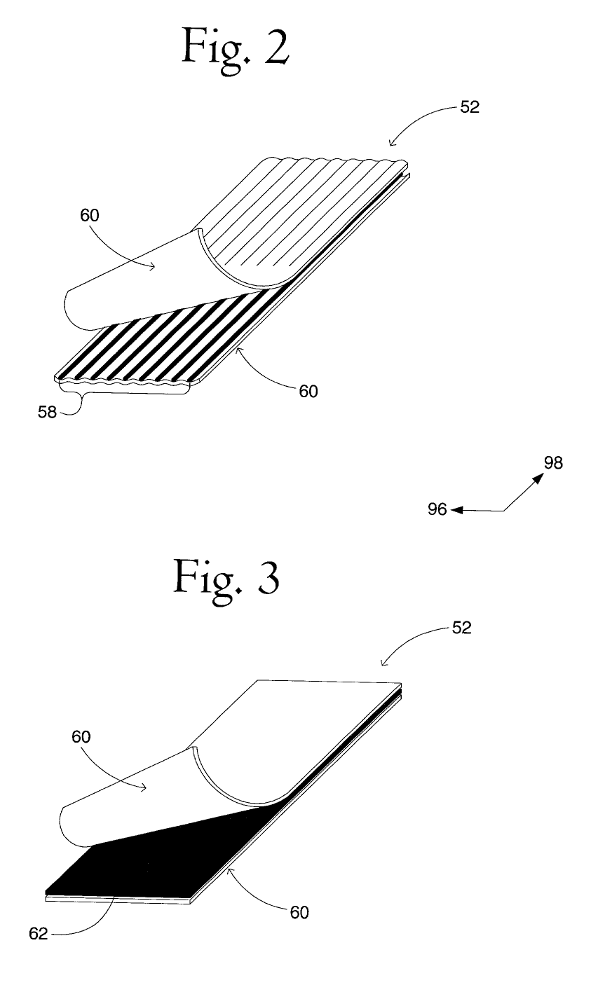

FIG. 2 illustrates an embodiment of a connector tab 52 in the fully-extended position. The tab 52 is comprised of a plurality of elastic elements 58 sandwiched between two layer elements 60. For clarity, one layer element is depicted after being partially peeled away from the plane of the tab. The layer elements can be made from a number of materials. In one embodiment the layer elements are made from spun-bonded nonwoven polypropylene material.

In the embodiment depicted in FIG. 2, the elastic elements 58 are strands of elastic material, oriented roughly parallel with the lateral axis 98 of the garment 20, which are attached to one or both of the layer elements 60 such that the layer elements 60 are shirred or gathered when the elastic elements 58 are in a relaxed or contracted position. The elastic elements 58 can be made from any number of elastic or elastomeric materials, such as rubber, spandex, or LYCRA. In a preferred embodiment, the elastic elements 58 are attached to the layer elements 60 using an elastic adhesive, and the layer elements 60 are attached to one another using the same elastic adhesive. The means of attachment preferably prevents the elastic elements 58 from becoming tangled or misaligned and maintains the wide flat shape of the tab 52, which ensures that the garment 20 is operative, comfortable, and appears tidy.

Although the above preferred embodiment is exemplary of one method of attaching the elastic elements 58 to the layer elements 60 and attaching the layer elements 60 to one another, it is anticipated that the elastic elements 58 can be attached by any number of means. In addition, the means of attachment may be either continuous throughout the entire area of the connector tabs 52, or intermittent and dispersed.

FIG. 3 illustrates a second embodiment of a tab 52 in the fully extended position. Again, for clarity, one layer element 60 is depicted after being partially peeled away from the plane of the tab. In this embodiment, a single elastic element 62 is sandwiched between two layer elements 60. The elastic element 62 can be comprised of a film of any suitable elastic material, including rubber, spandex, LYCRA and elastic polymers. The elastic element 62 also may be comprised of a multidirectional elastic aggregate such as elastic webbing, netting, or scrim elastic, such as FLEXCEL™ Elastic Nonwoven Laminate, available from Kimberly-Clark Corporation, headquartered in Neenah, Wis. The elastic element 62 can be attached to the layer elements 60 by any functional means, including elastic adhesives, as described above. Film, webbing, netting, and scrim elastic elements provide restoring forces in all planar directions, which may provide improved resistance to torsional forces in the plane of the garment 20. These torsional forces tend to cause the first waist edge 30 to move vertically relative to the second waist edge 32, making the garment 20 loose and uncomfortable, and allowing urine and BM leakage.

The connector tabs 52 also can be made from commercially available elastomeric materials such as Fabriflex®, available from Tredegar Film Products, headquartered in Richmond, Va. Such products are typically comprised of an elastic film layer sandwiched between nonwoven spun-bonded materials or similar constructions.

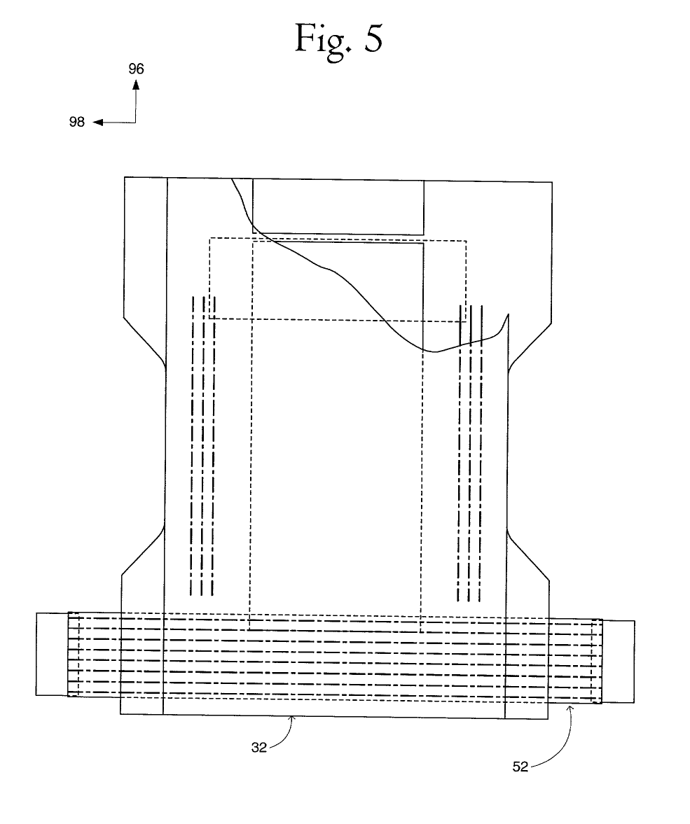

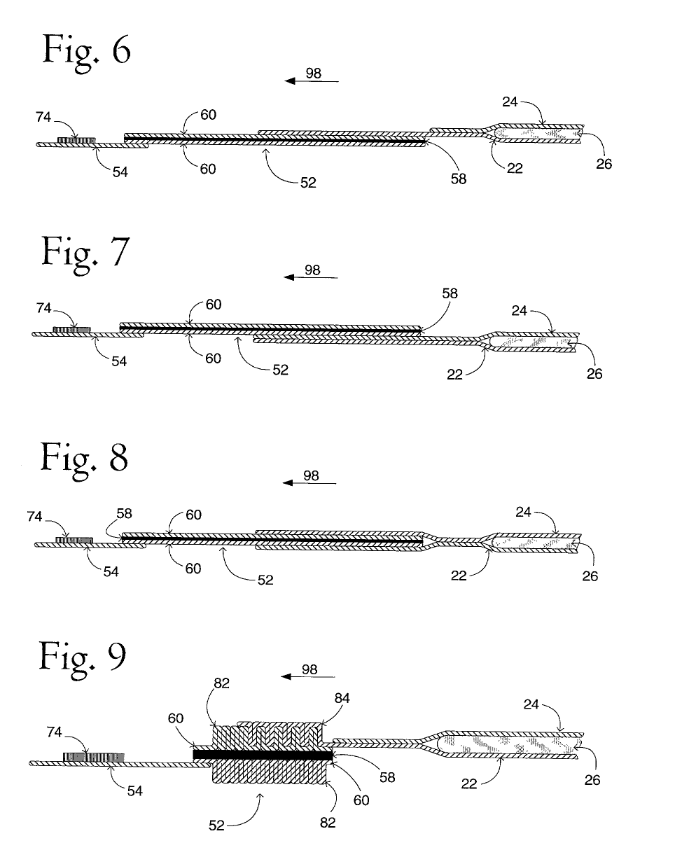

It is preferable to provide a single garment 20 that can be fitted to users with a variety of body sizes. Additionally, the garment's user or the user's caregiver should be able to unfasten and refasten the garment multiple times without reducing the utility of the garment. To meet these requirements, the tabs should be able to be extended and contracted many times without losing their elasticity. The elasticity is defined as the amount of deformation along the lateral axis of the garment (98 in FIGS. 6 and 7) that the tab 52 can experience without experiencing plastic deformation of the constituent parts or otherwise degrade the garment's ability to grip the user.

The elastic tabs 52 grip the user's torso and hold the garment in place. Ideally, the tabs will exhibit enough force to prevent the escape of bodily waste that is captured in the garment, but will also allow unrestricted movement and will not cause the user undue discomfort. The amount of gripping force exerted by the tabs is a function of the spring constant of the elastic tabs. The spring constant of the tabs 52 may be varied along the longitudinal dimension 96 or lateral dimension 98 of the tab, making the garment 20 more easily stretched in certain areas, which may be done to improve the fit, comfort, or sealing performance of the garment 20.

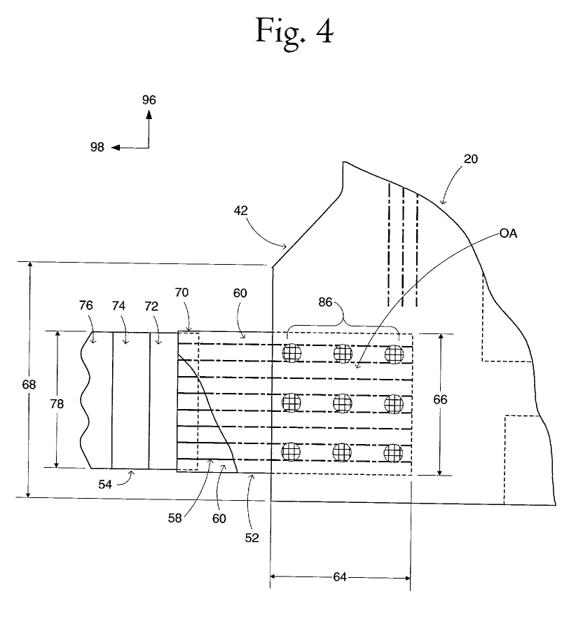

FIG. 4 is a partially-cutaway plan view of one embodiment of the invention in which the tab 52 is attached to the garment 20 at the second waist extension 42. In FIG. 4, the tab 52 is depicted partially cut away to show the elastic elements 58 sandwiched between the layer elements 60. The tab 52 is connected to the outer layer 22 in an overlap area OA defined by the tab overlap 64, which is in the lateral dimension 98 of the garment 20, and the tab width 66, which is in the longitudinal dimension 96 of the garment 20.

In a preferred embodiment, the tab 52 is attached to the garment 20 using construction adhesives, however, this attachment can be accomplished using any number of means well known in the art, such as ultrasonic bonding or pressure heat bonding. The use of construction adhesives is preferable because the construction adhesives are less likely to degrade the elasticity of the tabs 52. The connection or connections that join the tab 52 to the garment 20 can cover the entire overlap area OA, or they can be in a discrete area or areas within the overlap area OA. In the exemplary embodiment depicted in FIG. 4, a plurality of connection points 86 attach tab 52 to the garment 20. This exemplary embodiment should not be understood to limit the aforementioned variety of means for attaching the tab 52 to the garment 20.

In a preferred embodiment, the tab overlap distance 64 is greater than about 20 mm. The tab overlap distance 64 can be less than about 20 mm, but as this dimension decreases the beneficial attributes of the invention may decrease, and the strength of the attachment between the garment 20 and the tab 52 may decrease. The tab overlap distance 64 can be as great as one-half the lateral dimension 98 of the second waist edge 32.

Referring now to FIG. 5, in one embodiment of the invention, a single tab 52 traverses the entire width of the garment. In this embodiment of the invention, the full-width tab 52 may abrogate the need for a second waist elastic 50 at the second waist edge 32. The second waist elastic 50 may be retained in the garment, however, to provide improved fit and sealing characteristics.

Referring back to FIG. 4, in a preferred embodiment, the tab width 66 is greater than about 30 mm, and can be as great as the longitudinal dimension 96 of the second waist extension 42. A tab width 66 of less than 30 mm may be used, but it may be uncomfortable to the wearer, because it may concentrate the gripping force of the garment in a smaller area of the wearer's body. A narrower tab 52 also can have a weaker attachment to the garment 20 due to a decreased overlap area OA. Furthermore, a narrower tab 52 may be less able to resist torsional forces in the plane of the garment 20, as described before.

A gripping connector 54 is operably attached to the tab 52 at or near the tab's laterally outboard end 70. The gripping connector can be attached by any number of means well known in the art, such as construction adhesives, pressure heat bonding, or ultrasonic bonding. The gripping connector may be comprised of any of a multitude of fasteners well known in the art, such as hook and loop connectors, buttons, or snaps. In a preferred embodiment, the gripping connector 54 is comprised of a plurality of hook fasteners, and the target connector 56 is comprised of a plurality of loop connectors which interlock with the hook connectors. It is understood that the hook and loop connectors can be interposed, such that the gripping connector 54 is comprised of loop connectors, and the target connector 56 is comprised of corresponding interlocking hook connectors.

In the embodiment depicted in FIG. 4, the gripping connector 54 is comprised of a relief area 72 in which there are no connecting elements, a gripping area 74 that contains a plurality of connecting elements such as hook fasteners, and a grip area 76 that contains no connecting elements. The gripping connector 54 also may be constructed from a relatively rigid material so that it lies flat against the garment 20 when connected to the target connector 56, preventing inadvertent releases. The gripping connector width 78, which is in the longitudinal dimension 96 of the garment 20, can be substantially the same as the tab width 66 but there is no requirement that gripping connector width 78 be the same width as the tab width 66.

It is contemplated in the invention that the tab 52 can be attached to the garment 20 in a number of positions. FIG. 6 depicts an embodiment of the invention in which the tab 52 is located adjacent to the outer layer 22 of the garment 20. FIG. 7 depicts an embodiment of the invention in which the tab 52 is located adjacent to the inner layer 24 of the garment 20. FIG. 8 depicts an embodiment of the invention in which the tab 52 is sandwiched between the inner layer 24 and the outer layer 22 of the garment 20. In FIGS. 6, 7, and 8 the elastic elements 58 or elastic element 62 are depicted in the fully extended position. In addition to the aforementioned embodiments, other attachment positions are envisioned, and should be apparent to one skilled in the art. Similarly, it should be apparent that the gripping connector 54 can be attached to the tab 52 in a number of positions.

FIG. 9 is a cross-sectional view of the tab 52 attached adjacent to the outer layer 22 of a garment 20 as shown in FIG. 6. In FIG. 9 the elastic elements 58 or elastic element 62 are in the fully-relaxed position. In this position, the layer elements 60 of the tab 52 have shirrs or gathers 82. The outer layer 22 of the garment 20, to which the tab 52 is attached, also has shirrs or gathers 84 when the elastic elements 58 or elastic element 62 are relaxed. In this embodiment, both the tabs and the garment are expandable to fit the user.

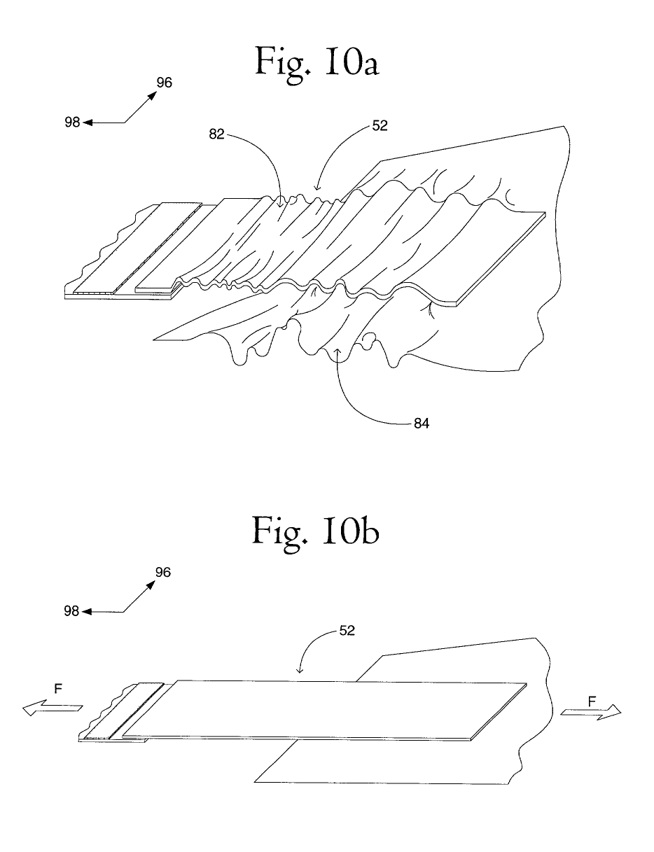

FIG. 10a is a drawing of the tab attached to the garment 20 with the elastic elements 58 or elastic element 62 in the relaxed position. The shirrs in the tab 82 and the shirrs in the garment 84 are visible in FIG. 10a. These shirrs 82, 84 expand to conform to and grip the body of the user.

FIG. 10b is a drawing of the tab attached to the garment 20 with the elastic elements 58 or elastic element 62 in the fully-extended position. The shirrs in the tab 82 and the shirrs in the garment 84, which were visible in FIG. 10a, have been eliminated by stretching the elastic of the tab 52 to its full length by applying opposing stretching forces F in the lateral dimension 98. The forces F are similar to the forces that are applied when fastening the garment 20 to the user. A restoring force, opposite in direction to the stretching forces F, is exerted by the elastic elements 58 or element 62 to return the tab 52 and garment 20 to the shirred or gathered state. This restoring force acts to contract the garment around the body of the user, holding the garment on the user, and preventing leakage of urine and BM from the waistline of the garment.

Referring back to FIG. 1, in a preferred embodiment, the connector tabs 52 and garment 20 may be designed to fit a variety of body sizes. When the gripping connectors 54 are attached to the target connector 56, the garment 20 encircles the waistline of the user. The body sizes that the garment 20 can fit are therefore a function of the distance between the ends of the gripping connectors 54 in the lateral dimension 98, and the width of the target connector 56. By increasing and decreasing the distance between the ends of the gripping connectors 54, the garment 20 can be made to fit around the circumference of various body sizes.

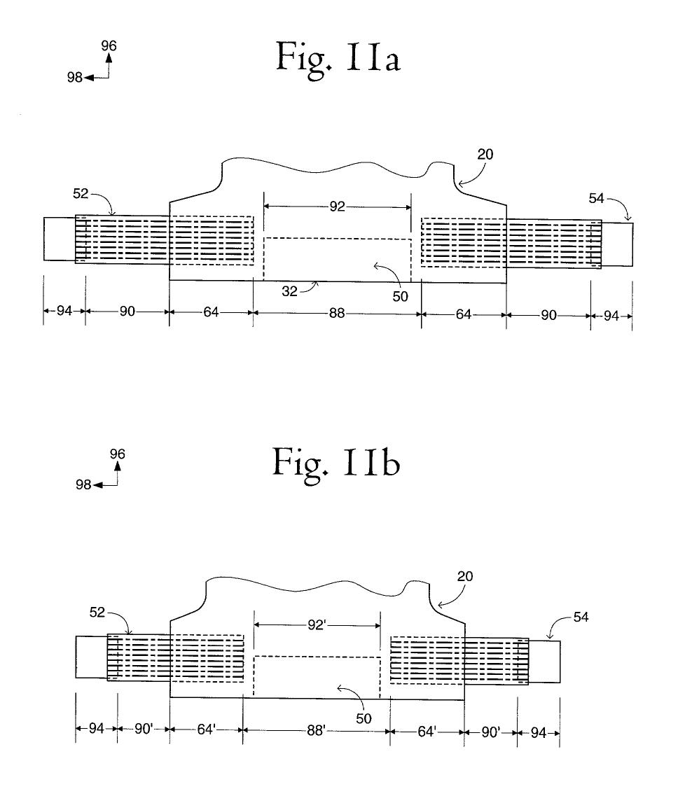

FIGS. 11a and 11b, are partial views of embodiments of the invention depicting the garment's 20 second waist edge 32, tabs 52, and second waist elastic 50. FIG. 11a depicts the embodiment in its fully extended position, and FIG. 11b depicts the embodiment in its fully contracted position.

Referring now to FIG. 11a, the maximum overall distance between the ends of the gripping connectors 54 is equal to the sum of the extended tab overlap 64, extended center width 88, extended tab reach 90, and gripping connector reach 94. The extended center width 88 is itself a function of the second waist elastic extended width 92. The extended tab overlap 64, and the contracted tab overlap 64′ can be referred to as the overlap distance. The extended tab reach 90, and the contracted tab reach 90′ can be referred to as the reach distance. It is preferred that the reach distance 90 is at least 10 mm, when tab 52 is fully extended. In FIG. 11b, the minimum overall distance between the ends of the gripping connectors 54 is equal to the sum of the contracted tab overlap 64′, contracted center width 88′, contracted tab reach 90′, and gripping connector reach 94. The contracted center width 88′ is itself a function of the second waist elastic contracted width 92′.

In one embodiment of the invention, the sum of the extended tab overlap 64 and the extended tab reach 90 is between approximately 5% and approximately 300% greater than the sum of the contracted tab overlap 64′ and the contracted tab reach 90′. In another embodiment of the invention, the sum of the extended tab overlap 64 and the extended tab reach 90 is between approximately 100% and approximately 250% greater than the sum of the contracted tab overlap 64′ and the contracted tab reach 90′. In yet another embodiment of the invention, the sum of the extended tab overlap 64 and the extended tab reach 90 is approximately 175% greater than the sum of the contracted tab overlap 64′ and the contracted tab reach 90′.

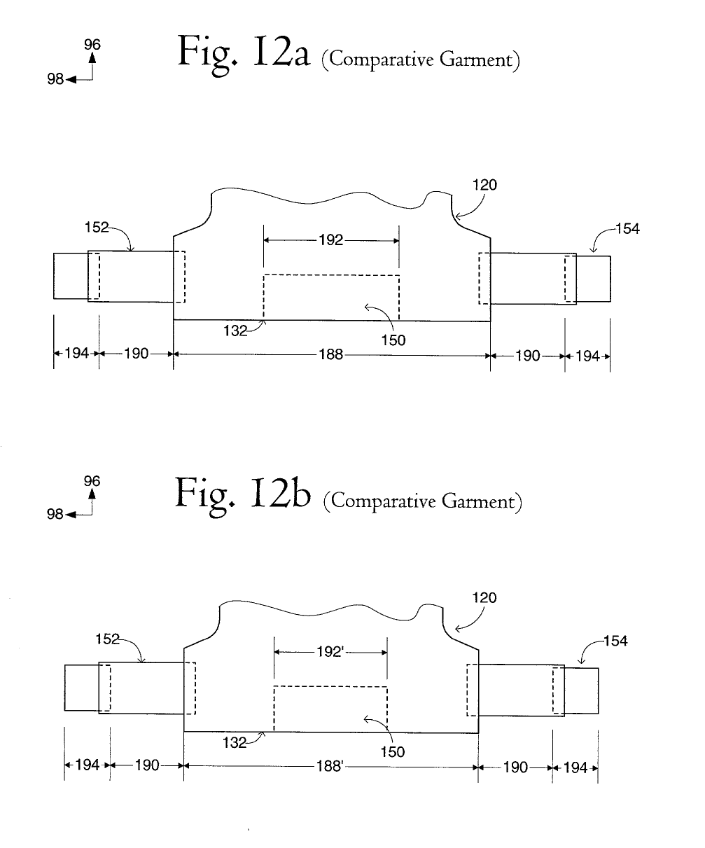

FIGS. 12a and 12b, are partial views of a comparative garment, depicting a typical garment's 120 second waist edge 132, tabs 152, gripping connectors 154 and second waist elastic 150. FIG. 12a depicts the comparative garment in its fully extended position, and FIG. 12b depicts the comparative garment in its fully contracted position.

FIG. 12a depicts a portion of a comparative garment 120 around the second waist edge 132, having a second waist elastic 150 and conventional non-elasticized connector tabs 152. The maximum distance between the ends of the gripping connectors 154 is the sum of the extended center width 188, the tab reach 190, and the gripping connector reach 194. In FIG. 12b the minimum distance between the ends of the gripping connectors 154 is the sum of the extended center width 188′, the tab reach 190, and the gripping connector reach 194.

A comparative garment 120 using conventional connector tabs 152, a conventional chassis, and conventional gripping connectors, has a maximum overall distance between the ends of the gripping connectors 154 of about 406 mm, and a minimum distance of about 380 mm. In an embodiment of the present invention, in which elasticized tabs are attached to the same conventional chassis and conventional gripping connectors as used in the comparative garment example above, the maximum overall distance between the ends of the gripping connectors 54 is about 470 mm, and the minimum distance is about 330 mm. Therefore, in this embodiment of the invention, the garment 20 can accommodate users with waists between 50 mm smaller and 64 mm larger than a conventional garment 120. It should be understood that this exemplary description of an embodiment of the invention should not be construed to limit the invention.

Other embodiments, uses, and advantages of the invention will be apparent to those skilled in the art from consideration of the specification and practice of the invention disclosed herein. The specification should be considered exemplary only, and the scope of the invention is accordingly intended to be limited to the following claims.

We claim:

1. An absorbent garment comprising:
an outer layer comprised of a liquid impervious material;
a liquid pervious inner layer;
an absorbent layer disposed between the inner and outer layers, the respective layers having opposed first and second laterally extending waist edges with a crotch region therebetween, the first and second waist edges for extending around the waist of a wearer, and two opposed side margins extending generally in a longitudinal direction between the first and second waist edges, and being narrower in at least a portion of the crotch region such that at least one of the outer layer and inner layer forms ears at the second waist edge;
a pair of elastically extensible connector tabs, each having a perimeter defined by a longitudinally extending inboard edge and a longitudinally extending outboard edge with laterally extending side edges therebetween, each inboard edge being located inboard of its respective side margin along the second waist edge by an overlap distance, and each inboard edge being located between the second waist edge and the crotch region, and each outboard edge being located outboard of its respective side margin by a reach distance;
the pair of elastically extensible connector tabs overlapping at least one of the outer layer and inner layer throughout respective overlap areas defined by each respective overlap distance and the longitudinally extending inboard edges of each respective connector tab;
the pair of elastically extensible connector tabs being operably connected to at least one of the outer layer and inner layer in each respective overlap area such that at least one of the outer layer and inner layer to which each connector tab is operably connected is shirred when the connector tab is not extended;
a gripping connector operably connected to each connector tab near the respective longitudinally extending outboard edge of each connector tab, and extending distally therefrom; and
one or more target connectors located along the first waist edge of the outer layer, the target connectors projecting outwardly from the outer layer and adapted each to receive at least one of the gripping connectors.

2. The absorbent garment as recited in claim 1, wherein each connector tab is comprised of a plurality of elastic elements disposed between layers of nonwoven fabric.

3. The absorbent garment as recited in claim 1, wherein each connector tab is comprised of an elastic film element disposed between layers of nonwoven fabric.

4. The absorbent garment as recited in claim 1, wherein each connector tab is comprised of a multidirectional aggregation of elastic elements disposed between layers of nonwoven fabric.

5. The absorbent garment as recited in claim 1, wherein the overlap distance is at least 20 mm when the elastically extensible connector tabs are fully extended.

6. The absorbent garment as recited in claim 1, wherein the reach distance is at least 10 mm when the elastically extensible connector tabs are fully extended.

7. The absorbent garment as recited in claim 1, wherein the longitudinally extending inboard edge has a longitudinal dimension of at least 30 mm.

8. The absorbent garment as recited in claim 1, wherein the longitudinally extending inboard edge has a longitudinal dimension substantially equal to the longitudinal dimension of the ears at the second waist edge.

9. The absorbent garment as recited in claim 1, wherein the connector tabs are approximately 175% longer in their fully extended position than in their relaxed position.

10. The absorbent garment as recited in claim 1, wherein the connector tabs have an elasticity between 5% and 300%.

11. The absorbent garment as recited in claim 1, wherein the connector tabs have a variable spring constant that varies along the lateral dimension of the connector tabs.

12. The absorbent garment as recited in claim 1, wherein the operable connection between the connector tabs and at least one of the outer layer and inner layer is comprised of a plurality of connection points distributed within the overlap area.

13. The absorbent garment as recited in claim 1, wherein the operable connection between the connector tabs and at least one of the outer layer and inner layer is comprised of a substantially continuous connection within the overlap area.

14. The absorbent garment recited in claim 1, wherein the connector tab is operably connected to the exterior surface of the outer layer.

15. The absorbent garment recited in claim 1, wherein the connector tab is operably connected to the exterior surface of the inner layer.

16. The absorbent garment recited in claim 1, wherein the connector tab is operable connected between the outer layer and the inner layer.

17. An absorbent garment comprising:
an outer layer comprised of a liquid impervious material;
a liquid pervious inner layer;
an absorbent layer disposed between the inner and outer layers, the respective layers having opposed first and second laterally extending waist edges with a crotch region therebetween, the first and second waist edges for extending around the waist of a wearer, and two opposed side margins extending generally in a longitudinal direction between the first and second waist edges, and being narrower in at least a portion of the crotch region such that at least one of the outer layer and inner layer forms ears at the second waist edge;
a single elastically extensible connector tab, having a pair of longitudinally extending outboard edges with laterally extending side edges therebetween, each longitudinally extending outboard edge being located outboard of each respective side margin along the second waist edge by a reach distance, [and the laterally extending side edges being located between the second waist edge and the crotch region];
the elastically extensible connector tab overlapping, in a planar sense, at least one of the outer layer and inner layer throughout an overlap area defined by the side margins and the laterally extending side edges;
the elastically extensible connector tab being operably connected to at least one of the outer layer and inner layer in the overlap area such that at least one of the outer layer and inner layer to which the connector tab is operably connected is shirred when the connector tab is not extended;
a gripping connector operably connected to the connector tab near each respective longitudinally extending outboard edge of the connector tab, and extending distally therefrom; and
target connectors located along the first waist edge of the outer layer, the target connectors projecting outwardly from the outer layer and adapted each to receive at least one of the gripping connectors.

18. The absorbent garment as recited in claim 17, wherein the connector tab is comprised of a plurality of elastic elements disposed between layers of nonwoven fabric.

19. The absorbent garment as recited in claim 17, wherein the connector tab is comprised of an elastic film element disposed between layers of nonwoven fabric.

20. The absorbent garment as recited in claim 17, wherein the connector tab is comprised of a multidirectional aggregation of elastic elements disposed between layers of nonwoven fabric.

21. The absorbent garment as recited in claim 17, wherein the reach distance is at least 10 mm when the elastically extensible connector tab is fully extended.

22. The absorbent garment as recited in claim 17, wherein the longitudinally extending inboard edge has a longitudinal dimension of at least 30 mm.

23. The absorbent garment as recited in claim 17, wherein the longitudinally extending inboard edge has a longitudinal dimension substantially equal to the longitudinal dimension of the ears at the second waist edge.

24. The absorbent garment as recited in claim 17, wherein the connector tab is approximately 175% longer in its fully extended position than in the relaxed position.

25. The absorbent garment as recited in claim 17, wherein the connector tab has an elasticity between 5% and 300%.

26. The absorbent garment as recited in claim 17, wherein the connector tab has a variable spring constant that varies along the lateral dimension of the connector tab.

27. The absorbent garment as recited in claim 17, wherein the operable connection between the connector tab and at least one of the outer layer and inner layer is comprised of a plurality of connection points distributed within the overlap area.

28. The absorbent garment as recited in claim 17, wherein the operable connection between the connector tab and at least one of the outer layer and inner layer is comprised of a substantially continuous connection within the overlap area.

29. The absorbent garment recited in claim 17, wherein the connector tab is operably connected to the exterior surface of the outer layer.

30. The absorbent garment recited in claim 17, wherein the connector tab is operably connected to the exterior surface of the inner layer.

31. The absorbent garment recited in claim 17, wherein the connector tab is operably connected between the outer layer and the inner layer.
